(19) United States
(12) Patent Application Publication
(10) Pub. No.: US 2013/0030243 A1
(43) Pub. Date: Jan. 31, 2013

(54) ANTI-BACTERIAL SURGICAL MESHES

(71) Applicants: Mark Boden, Harrisville, RI (US); Steve Kangas, Woodbury, MN (US); Jeri'Ann Hiller, Westford, MA (US); Bruce Forsyth, Hanover, MN (US)

(72) Inventors: Mark Boden, Harrisville, RI (US); Steve Kangas, Woodbury, MN (US); Jeri'Ann Hiller, Westford, MA (US); Bruce Forsyth, Hanover, MN (US)

(73) Assignee: Boston Scientific Scimed, Inc., Maple Grove, MN (US)

(21) Appl. No.: 13/558,409

(22) Filed: Jul. 26, 2012

Related U.S. Application Data
(60) Provisional application No. 61/512,699, filed on Jul. 28, 2011.

(51) Int. Cl. A61F 2/02; B05D 5/00

(52) U.S. Cl. 600/37; 427/2.24

(57) ABSTRACT

According to an aspect of the present invention, surgical meshes are provided which release one or more antimicrobial agents in an amount sufficient to reduce the risk of microbial infection upon implantation of the mesh. Other aspects of the invention pertain to methods of making and using such surgical meshes.

### STATEMENT OF RELATED APPLICATION

This application claims the benefit of U.S. Ser. No. 61/512,699, filed Jul. 28, 2011 and entitled “ANTI-BACTERIAL SURGICAL MESHES,” which is hereby incorporated by reference in its entirety.

### FIELD OF THE INVENTION

The present invention relates generally to medical articles, and more particularly to surgical meshes.

### BACKGROUND OF THE INVENTION

Pelvic floor (pelvic support) disorders involve a dropping down (prolapse) of the bladder, rectum, or uterus caused by weakness of or injury to the ligaments, connective tissue, and muscles of the pelvis. The different types of pelvic floor disorders are named according to the organ affected. For example, a rectocele develops when the rectum drops down and protrudes into the back wall of the vagina. An enterocele develops when the small intestine and the lining of the abdominal cavity (peritoneum) bulge downward between the uterus and the rectum or, if the uterus has been removed, between the bladder and the rectum. A cystocele develops when the bladder drops down and protrudes into the front wall of the vagina. In prolapse of the uterus (procidentia), the uterus drops down into the vagina. See, e.g., The Merck Manuals Online Medical Library, Home Edition, “Pelvic Floor Disorders.” Pelvic floor disorders are commonly treated by implanting a surgical mesh within the patient's pelvis to support the organ or organs that require support.

Urinary incontinence affects millions of men and women of all ages in the United States. Stress urinary incontinence (SUI) affects primarily women and is generally caused by two conditions, intrinsic sphincter deficiency (ISD) and hypermobility. These conditions may occur independently or in combination. In ISD, the urinary sphincter valve, located within the urethra, fails to close properly (coapt), causing urine to leak out of the urethra during stressful activity. Hypermobility is a condition in which the pelvic floor is distended, weakened, or damaged, causing the bladder neck and proximal urethra to rotate and descend in response to increases in intra-abdominal pressure (e.g., due to sneezing, coughing, straining, etc.). The result is that there is an insufficient response time to promote urethral closure and, consequently, urine leakage and/or flow results. A popular treatment of SUI is via the use of a surgical mesh, commonly referred to as a sling, which is permanently placed under a patient's bladder neck or mid-urethra to provide a urethral platform. Placement of the sling limits the endopelvic fascia drop, while providing compression to the urethral sphincter to improve coaptation. Further information regarding sling procedures may be found, for example, in the following: Fred E. Govier et al., “Pubocaginal slings: a review of the technical variables,” Curr. Opin. Urol. 11:405-410, 2001, John Klutke and Carl Klutke, “The promise of tension-free vaginal tape for female SUI,” Contemporary Urol. pp. 59-73, October 2000; and PCT Patent Publication No. WO 00/74633 A2: “Method and Apparatus for Adjusting Flexible Areal Polymer Implants.”

Further uses of surgically implantable meshes include meshes for hernia repair (e.g., meshes for inguinal hernia, hiatus hernia, etc.), meshes for thoracic wall defects, breast support meshes and various other soft-tissue surgical mesh support devices, including meshes for cosmetic and reconstructive surgery, among others.

### SUMMARY OF THE INVENTION

According to an aspect of the present invention, surgical meshes are provided which release one or more antimicrobial agents in an amount sufficient to reduce the risk of microbial infection upon implantation of the mesh.

Other aspects of the invention pertain to methods of making and using such surgical meshes.

These and other aspects, embodiments and advantages of the present invention will become immediately apparent to those of ordinary skill in the art upon review of the Detailed Description and any claims to follow.

### BRIEF DESCRIPTION OF THE DRAWING

FIG. 1 is a schematic top view of a surgical mesh, in accordance with an embodiment of the invention.

FIG. 2 is a schematic top view of a surgical mesh, in accordance with another embodiment of the invention.

FIG. 3 is a schematic top view of a surgical mesh, in accordance with yet another embodiment of the invention.

### DETAILED DESCRIPTION OF THE INVENTION

A more complete understanding of the present invention is available by reference to the following detailed description of numerous aspects and embodiments of the invention. The detailed description of the invention which follows is intended to illustrate but not limit the invention.

The meshes described herein are typically “substantially two-dimensional” in shape. As used herein a “substantially two-dimensional” object is a sheet-like object, typically one whose length and width are at least 10 times greater than the thickness of the material forming the object, for example, whose length and width are each 10 to 25 to 50 to 100 or more times the thickness. For example, surgical meshes may be in the form of ribbons, sheets, and other more complex sheet-like shapes (see, e.g., FIGS. 1 to 3 below). In certain embodiments, the mesh will be able to take on a planar configuration, for example, when placed on a planar surface such as a table top. However, substantially two-dimensional objects, including the surgical meshes of the invention, need not be planar. For example, such objects may curve in space (e.g., as a substantially two-dimensional orange peel curves around the inner portion of the orange, etc.).

Surgical meshes in accordance with the present invention may be in the form of ribbons, sheets, and other more complex sheet-based shapes.

Surgical meshes in accordance with the present invention are typically formed using one or more filaments (e.g., fibers, fibrils, threads, yarns, etc.). Thus, surgical meshes in accordance with the present invention include monofilament and multifilament meshes. Surgical meshes in accordance with the present invention include woven meshes and non-woven meshes (including knitted meshes, felt meshes, and spunbound meshes, among others).

Surgical meshes in accordance with the present invention include meshes having small pores (less than 1 mm) and those having large pores (greater than or equal to 1 mm). In various embodiments, the surgical meshes of the invention preferably have pore sizes ranging from 10 μm to 50 μm to 100 μm to 0.5 mm to 1 mm to 5 mm to 10 mm to 50 mm to 100 mm in diameter, more preferably 0.5 mm to 10 mm in certain embodiments. The pore size can be varied prevent or promote tissue in-growth.

Filament(s) for the surgical meshes of the present invention preferably range from 1 μm to 5 μm to 10 μm to 50 μm to 100 μm to 500 μm to 1 mm in diameter, more preferably from 50 μm to 500 μm in diameter, in certain embodiments.

Surgical meshes in accordance with the invention include, for example, meshes for pelvic floor repair, meshes for renal pelvis repair, urethral slings, vaginal slings, hernia meshes (e.g., meshes for inguinal hernia, hiatus hernia, etc.), meshes for thoracic wall defects, breast support meshes and various other soft-tissue surgical mesh support devices, including meshes for cosmetic and reconstructive surgery, among others. Surgical meshes may be surgically implanted in a variety of subjects, typically vertebrate subjects, more typically mammalian subjects, including human subjects, pets and livestock.

Filaments for forming meshes in accordance with the present invention include those formed from various synthetic biostable or biodisintegrable polymers, various naturally occurring polymers, as well as various biologics.

Examples of synthetic biostable polymers may be selected from the following: (a) polyolefin homopolymers and copolymers, including homopolymers and copolymers of C2-C8 alkenes, for example, polyethylene and polypropylene among others, (b) fluoropolymers, including homopolymers and copolymers of C2-C8 alkenes in which one or more hydrogen atoms are substituted with fluorine, for example, polytetrafluoroethylene (PTFE), polyvinylidene fluoride (PVDF), poly(vinylidene fluoride-co-hexafluoropropene) (PVDF-HFP) among others, (c) polyamides such as nylons, among others, (d) polyesters, including, for example, polyethylene terephthalate, among others, (e) styrenic copolymers such as isobutylene-styrene copolymers, including block copolymers comprising one or more polystyrene blocks and one or more polyisobutylene blocks, for instance, poly(styrene-b-isobutylene-b-styrene) (SIBS), among others, (e) polyurethanes such as polyisobutylene based polyurethanes (PIB-PU), among others, (f) as well as various other non-absorbable polymers.

Where isobutylene-styrene copolymers (e.g., SIBS) are used, the ratio of monomers in these polymers can be selected to obtain mechanical properties such that tissue compatibility is enhanced. For example, a higher isobutylene content will result in a softer polymer that may be a better match for the durometer of the surrounding tissue.

Examples of synthetic biodegradable polymers may be selected, for example, from polyesters and polyanhydrides, among others. Specific biodegradable polymers may be selected from suitable members of the following, among others: (a) polyester homopolymers and copolymers (including polyesters and poly[ester-amides]), such as polyglycolide, polylactide (PLA), including poly-L-lactide, poly-D-lactide, and poly-D,L-lactide, poly(lactide-co-glycolide) (PLG), including poly(L-lactide-co-glycolide), poly(D-lactide-co-glycolide) and poly(D,L-lactide-co-glycolide), poly(beta-hydroxybutyrate), poly-D-gluconate, poly-L-gluconate, poly-D,L-gluconate, poly(epsilon-caprolactone), poly(delta-valerolactone), poly(p-dioxanone), poly(trimethylene carbonate), poly(lactide-co-delta-valerolactone), poly(lactide-co-epsilon-caprolactone), poly(lactide-co-beta-malic acid), poly(lactide-co-trimethylene carbonate), poly(glycolide-co-trimethylene carbonate), poly(beta-hydroxybutyrate-co-beta-hydroxyvalerate), poly[1,3-bis(p-carboxyphenoxy)propane-co-sebacic acid], poly(sebacic acid-co-fumaric acid), and poly(ortho esters) such as those synthesized by copolymerization of various diketene acetals and diols, among others; and (b) polyanhydride homopolymers and copolymers such as poly(adipic anhydride), poly(suberic anhydride), poly(sebacic anhydride), poly(dodecanedioic anhydride), poly(maleic anhydride), poly[1,3-bis(p-carboxyphenoxy)methane anhydride], and poly[alpha,omega-bis(p-carboxyphenoxy)alkane anhydrides] such as poly[1,3-bis(p-carboxyphenoxy)propane anhydride] and poly[1,3-bis(p-carboxyphenoxy)hexane anhydride], among others.

Where copolymers are employed, copolymers with a variety of monomer ratios may be available. For example, where PLG is used to form the microparticles, a variety of lactide:glycolide molar ratios will find use herein, and the ratio is largely a matter of choice, depending in part on any coadministered adsorbed and/or entrapped species and the rate of degradation desired. For example, a 50:50 PLG polymer, containing 50% D,L-lactide and 50% glycolide, will provide a faster resorbing copolymer, while 75:25 PLG degrades more slowly, and 85:15 and 90:10, even more slowly, due to the increased lactide component. Degradation rate can also be controlled by such factors as polymer molecular weight and polymer crystallinity.

More broadly, where used, PLG copolymers include those having a lactide/glycolide molar ratio ranging, for example, from 10:90 or less to 15:85 to 20:80 to 25:75 to 40:60 to 45:55 to 50:50 to 55:45 to 60:40 to 75:25 to 80:20 to 85:15 to 90:10 or more, and having a molecular weight ranging, for example, from 5,000 or less to 10,000 to 20,000 to 40,000 to 50,000 to 70,000 to 100,000 to 200,00 or more Daltons.

Where a biodegradable polyester is used (e.g., PLA, PLGA, etc.), one or more soft blocks (e.g., polyethylene oxide, polycaprolactone, etc.) may be included with one or more polyester blocks in the polymer to vary hardness, elongation, and degradation rate of the polymer.

Examples of naturally occurring polymers include biostable and biodegradable polymers such as cellulose, biocellulose, and alginates (non crosslinked and ionically crosslinked).

As defined herein, a “biologic material” is a material that comprises one or more extracellular matrix components. Biologic materials for use herein include crosslinked and non-crosslinked allograft (e.g., human cadaveric) materials, as well as crosslinked and non-crosslinked heterograft (e.g., bovine, porcine, equine, etc.) materials. Specific examples of non-crosslinked biologic materials include mammalian non-crosslinked biologic matrix materials, such as human dermis, human fascia lata, fetal bovine dermis and porcine small intestinal submucosa. Specific examples of crosslinked biologic materials include mammalian crosslinked biologic materials such as crosslinked porcine dermis, crosslinked porcine small intestinal submucosa, crosslinked bovine pericardium, and crosslinked horse pericardium. Such materials are typically acellular. Moreover, they are typically predominantly collagen.

In various embodiments, the meshes of the present invention comprise one or more antimicrobial agents, more preferably, one or more antibiotic agents. Such substances are provided, for example, to reduce the risk of microbial infection (including biofouling of the mesh and infection in surrounding tissue) upon implantation of the mesh, among other purposes.

Mesh erosion into the vagina, rectum or other organs is a rare, but significant complication from mesh-based treatments, including pelvic organ prolapse repair. Erosions can be influenced by a number of factors, including pore size, monofilament vs. multifilament mesh, scarring (shrinkage), mesh degradation, and infection. Infection can be particularly damaging. The surgical environment is often difficult to keep clean, and as a result, bacteria can be introduced to the site. There is a potential for the bacteria to lie dormant for some period of time, encased in a slime layer that protects it from initial antibiotic treatment. In addition, the mesh may not be well vascularized, so subsequent systemic treatment may not be effective. The initial response to infection can produce erosions due to inflammation. Also, certain polymers such as polypropylene are susceptible to reactive oxygen species which are generated by macrophages. The resultant reactions can embrittle the mesh, making it more likely to fail or cause tissue damage.

Accordingly, infection control is a desired step in mesh-based treatments, including pelvic organ prolapse repair. Antibiotic selection typically addresses the specific bacteria that are most commonly found at the implantation site. Moreover, the duration of activity may be gauged to insure that dormant infections are addressed.

Antibiotic agents may be selected, for example, from one or more of the following: the penicillins (e.g., penicillin G, methicillin, oxacillin, ampicillin, amoxicillin, ticarcillin, etc.), the cephalosporins (e.g., cephalothin, cefazolin, cefoxitin, cefotaxime, cefaclor, cefoperazone, cefixime, ceftriaxone, cefuroxime, etc.), the carbapenems (e.g., imipenem, metropenem, etc.), the monobactems (e.g., aztreonem, etc.), the carbacephems (e.g., loracarbef, etc.), the glycopeptides (e.g., vancomycin, teichoplanin, etc.), bacitracin, polymyxins, colistins, fluoroquinolones (e.g., norfloxacin, lomefloxacin, fleroxacin, ciprofloxacin, enoxacin, trovafloxacin, gatifloxacin, etc.), sulfonamides (e.g., sulfamethoxazole, sulfanilamide, etc.), diaminopyrimidines (e.g., trimethoprim, etc.), rifampin, aminoglycosides (e.g., streptomycin, neomycin, netilmicin, tobramycin, gentamicin, amikacin, etc.), tetracyclines (e.g., tetracycline, doxycycline, demeclocycline, minocycline, etc.), spectinomycin, macrolides (e.g., erythromycin, azithromycin, clarithromycin, dirithromycin, troleandomycin, etc.), and oxazolidinones (e.g., linezolid, etc.), among others, as well as pharmaceutically acceptable salts, esters and other derivatives of the same.

In some embodiments, any two of the above antibiotics may be employed in ratios (wt:wt) ranging from 5:95 or less to 10:90 to 15:85 to 20:80 to 25:75 to 30:70 to 40:60 to 50:50 to 60:40 to 70:30 to 75:25 to 80:20 to 85:15 to 90:10 to 95:5 or more.

In some embodiments, meshes can be prepared with an antibiotic cocktail that will be effective against skin-borne pathogens, particularly Staphylococcus aureus (S. aureus), including Methicillin-resistant Staphylococcus aureus (MRSA), and Escherichia coli (E. coli), reducing the risk of infection (including infection by such organisms) upon implantation of the mesh, for example, by a factor of 2 to 4 to 10 times or more. One antibiotic combination that has proven extremely effective against a broad spectrum of skin-borne pathogens is minocycline and rifampin. Other common antibiotics (e.g., gentamicin, tetracyclines, etc.) can also be used, either alone or in combination to address such pathogens.

Antibiotic agents may be associated with the meshes in various ways, including the following, among others: (a) loaded in the interior (bulk) of the filament(s), (b) bound to the surface of the filament(s) by covalent interactions and/or non-covalent interactions (e.g., interactions such as van der Waals forces, hydrophobic interactions and/or electrostatic interactions, for instance, charge-charge interactions, charge-dipole interactions, and dipole-dipole interactions, including hydrogen bonding), (c) applied as a coating (biostable or biodegradable) that at least partially surrounds the filament(s), and (d) combinations of the forgoing.

In various embodiments, at least one antibiotic agent is provided within a polymeric matrix. In some of these embodiments, the polymeric matrix corresponds to a bulk filament. In others of these embodiments, the polymeric matrix corresponds to a coating on a filament.

In various embodiments, the polymeric matrix (bulk filament, coating, etc.) contains 1 wt % or more of one or more antibiotic agents (e.g., from 1 wt % to 2 wt % to 5 wt % to 10 wt % to 25 wt % to 40 wt % to 50 wt % to 60 wt % to 70 wt % to 80 wt % to 90 wt % to 95 wt % to 98 wt % to 99 wt % or more). More typically, the polymeric matrix is a coating that contains 10 to 75 wt % of one or more antibiotic agents.

Meshes in accordance with the present disclosure preferably contain from 1 to 200 g of one or more antibiotic agents per m² of mesh area (e.g., from 1 to 2 to 50 to 10 to 25 to 50 to 100 to 200 g/m²), more preferably from 5 to 25 g/m².

For example, a mesh that is 5 cm wide and 20 cm long has a mesh area of 100 cm² or 0.01 m². If such a mesh were to be loaded with a total of 0.25 g of one or more antibiotic agents, that mesh would have an antibiotic loading of 0.25 g÷0.01 m²=25 g/m².

Coatings in accordance with the invention may contain one or more polymers, which may be selected, for example, from synthetic biostable or biodisintegrable polymers, naturally occurring polymers, and biologics, specific examples of which are set forth above, as well as additional polymers such as poly(vinyl alcohol) (PVA) and ethylene vinyl acetate copolymers (EVA), among others. Coatings may also contain one or more non-polymeric matrix materials, such as trehalose, fatty acids, triglycerides, iopromide, etc.

Biodisintegrable coatings may have an added benefit, for example, of ensuring essentially 100% release of any antibiotic therein. Biostable coatings, on the other hand, may have an added benefit, for example, of protecting an underlying mesh material (e.g., from oxidation, etc.).

Typical drug-to-polymer ratios (wt:wt) within the polymeric matrices (e.g., coatings, bulk filaments, etc.) range from 5:95 or less to 10:90 to 15:85 to 20:80 to 25:75 to 30:70 to 40:60 to 50:50 to 75:25 or more.

The amount and type of polymer that is selected will control the dose and duration of release.

Typical coating thicknesses range from 1 micrometer (micron) or less to 15 microns or more (e.g., from 1 to 2 to 5 to 7.5 to 10 to 12.5 to 15 microns in thickness), among other values.

As noted above, filaments in certain embodiments are not coated. For example, a filament may be spun (e.g., melt spun, wet spun, dry spun, electrospun, etc.) from a mixture comprising polymer(s), antibiotic agent(s), optional additional materials, solvents (if needed), and so forth. The resulting antibiotic-agent-containing filament may then serve both a structural (e.g., load bearing) function and a drug release function. Alternatively, the resulting antibiotic-agent-containing filament may be combined (e.g., woven into a mesh, etc.) with structural filaments that do not contain an antibiotic agent. Antibiotic-agent-containing filaments and structural filaments may be formed using one or more biostable or biodegradable polymers, selected, for example, from those set forth above.

Meshes in accordance with the invention may also comprise materials in addition to one or more polymers and one or more antibiotic agents. Such additional materials may be selected, for example, from additional therapeutic agents, plasticizers and fillers.

Examples of additional therapeutic agents include the following, among many others: (a) anti-inflammatory agents (e.g., for purposes of reducing macrophage levels, resulting in less muscle regeneration and re-growth) including corticosteroids such as hydrocortisone and prednisolone, and non-steroidal anti-inflammatory drugs (NSAIDS) such as aspirin, ibuprofen, and naproxen; (b) narcotic and non-narcotic analgesics and local anesthetic agents (e.g., for purposes of minimizing pain); (c) growth factors such as epidermal growth factor and transforming growth factor-α (e.g., for purposes of stimulate the healing process and or promoting growth of collagenous tissue); and (d) combinations of the foregoing.

The additional therapeutic agents may be associated with the meshes in various ways, including those described above in conjunction with antibiotic agents.

Examples of plasticizers may be selected from one or more of the following organic plasticizers, among others: citrate esters such as tributyl, triethyl, triacetyl, acetyl triethyl, and acetyl tributyl citrate (ATBC), dioxane, phthalate derivatives such as dimethyl, diethyl and dibutyl phthalate, glycerol, glycols such as polypropylene, propylene, polyethylene and ethylene glycol, surfactants such as sodium dodecyl sulfate and polyoxymethylene (20) sorbitan and polyoxyethylene (20) sorbitan monooleate. Such plasticizers may be provided for better handling and a more robust polymeric matrix (e.g., bulk filament, coating, etc.).

Meshes in accordance with the present disclosure may be provided in a wide range of shapes and sizes.

There is schematically illustrated in FIG. 1, a mesh 100, such as a urethral sling. The material for the mesh 100 comprises antibiotic-containing filament (e.g., coated or uncoated) as described above. Typical dimensions for such a urethral sling range from 1 to 25 cm (e.g., 1 to 2 to 5 to 10 to 20 to 25 cm) in length and from 1 to 25 cm (e.g., 1 to 2 to 5 to 10 to 20 to 25 cm) in width.

As previously noted, pelvic floor (pelvic support) disorders involve a dropping down (prolapse) of the bladder, rectum and/or uterus caused by weakness of or injury to the ligaments, connective tissue, and muscles of the pelvis. As with SUI, treatment of pelvic floor disorders may be treated by implanting a surgical mesh within the patient's pelvis to support the organ or organs that require support.

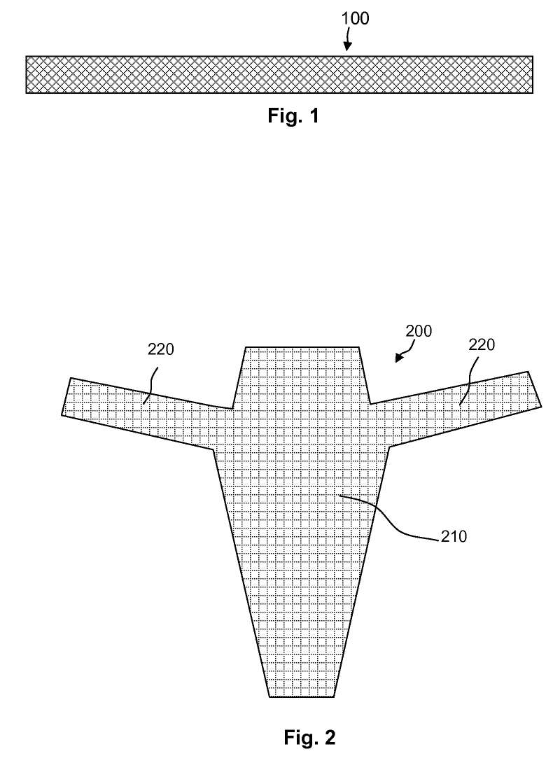

In accordance with one embodiment, there is schematically illustrated in FIG. 2 a mesh 200, for example, a pelvic floor repair mesh, having a central portion 210 and a plurality of arms 220 that emanate from the central portion 210. As used herein an “arm” is an elongated mesh component whose length is at least two times greater than its width, typically ranging from 2 to 4 to 6 to 8 to 10 or more times the width. (Thus, surgical sling 100 of FIG. 1 can be thought of as a single-arm device.) The material for the mesh 200 comprises antibiotic-containing filament (e.g., coated or uncoated) as described above.

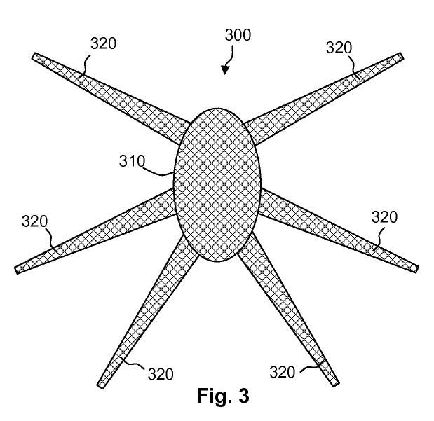

Although the mesh of FIG. 2 has two rectangular arms and a polygonal central body portion, other body and arm shapes and other numbers of arms may be used (e.g., 3, 4, 5, 6, 7, 8, etc.). As one specific variation, FIG. 3 illustrates a mesh 300 having a non-circular (oval) central body portion 310 and six non-rectangular (trapezoidal) arms 320, among near-limitless other possibilities. The material for the mesh 300 comprises antibiotic-containing filament (e.g., coated or uncoated) as described above.

Other aspects of the present disclosure pertain to methods by which surgical meshes may be created.

In certain embodiments, filaments and filament coatings can be formed from fluids that comprise at least one type of polymer, at least one type of antibiotic agent and, optionally, one or more additional materials (e.g., additional therapeutic agents, plasticizers, etc.).

For example, thermoplastic processing techniques may be used to form filaments and filament coatings. For instance, filaments can be spun from a melt that contains one or more polymers, one or more antibiotic agents and, optionally, one or more additional materials (e.g., via melt spinning), and (b) subsequently cooling the melt. Similarly, coatings can be formed, for instance, by (a) applying to a filament (or a surgical mesh formed from one or more filaments) a melt that contains one or more polymers, one or more antibiotic agents and, optionally, one or more additional materials, and (b) subsequently cooling the melt.

As another example, solvent-based techniques may be used to form filaments and filament coatings. For instance, filaments can be spun from a solution or dispersion that contains one or more solvent species, one or more polymers, one or more antibiotic agents and, optionally, one or more additional materials (e.g., via dry spinning, electrospinning, etc.), and (b) subsequently removing the solvent species. Similarly, coatings can be formed by (a) applying to a filament (or a surgical mesh formed from one or more filaments) a solution or dispersion that contains one or more solvent species, one or more polymers, one or more antibiotic agents and, optionally, one or more additional materials, and (b) subsequently removing the solvent species. A residual amount of solvent (e.g., less than 1 wt %, less than 0.1 wt %, less than 0.01 wt %, less than 0.001 wt %, or even less) may remain in the filament and/or coating.

In certain beneficial embodiments, a coating containing one or more polymers (e.g., PLGA, etc.), one or more antibiotics (e.g., rifampin and minocycline, etc.) and a solvent system of one or more solvents (e.g., N,N-dimethylformamide and tetrahydrofuran, etc.) is applied to a suitable mesh material (e.g., polypropylene mesh, etc.). The solvent system is capable of dissolving the polymer and the antibiotics and is compatible (e.g., does not dissolve) the mesh material. Solids content may vary widely, with low solids (e.g., 1 to 8 wt %) being preferred in some embodiments. Adhesion may be improved in some embodiments by cleaning the mesh with a suitable detergent, washing the mesh, or etching the mesh with a suitable plasma.

In some embodiments, coatings in accordance with the invention are applied in the form of a fluid (e.g., a solution, dispersion, melt, etc.) using a suitable application technique, which may be selected, for example, from dipping techniques, spraying techniques, spin coating techniques, web coating techniques, techniques involving coating via mechanical suspension including air suspension, electrostatic techniques, techniques in which fluid is selectively applied to only to certain regions of the mesh, for example, through the use of a suitable application device such as a sprayer, brush, roller, pen, or printer (e.g., screen printing device, ink jet printer, etc.). Suitable spray systems can be found, for example, in U.S. Pat. Nos. 6,861,088, 6,743,463, 6,669,980, 6,156,373, 6,322,847, 6,120,536 and 5,980,972.

Various aspects of the invention of the invention relating to the above are enumerated in the following paragraphs:

Aspect 1. A surgical mesh comprising at least one antimicrobial agent in an amount sufficient to reduce the risk of infection of the mesh by Staphylococcus aureus.

Aspect 2. The surgical mesh of aspect 1, wherein the at least one antimicrobial agent comprises a rifamycin group antibiotic, a tetracycline antibiotic, or a combination thereof.

Aspect 3. The surgical mesh of aspect 1, wherein the at least one antimicrobial agent comprises minocycline and rifampin as antibiotics.

Aspect 4. The surgical mesh of aspect 3, wherein aminocycline:rifampin weight ratio ranges from 1:10 to 2:1.

Aspect 5. The surgical mesh of aspect 3, wherein the total antibiotic concentration ranges from 5 to 100 grams of antibiotics per square meter of mesh.

Aspect 6. The surgical mesh of aspect 1, comprising a core comprising a first polymer and a coating comprising a second polymer and said at least one antimicrobial agent.

Aspect 7. The surgical mesh of aspect 6, wherein the first polymer is a biostable polymer.

Aspect 8. The surgical mesh of aspect 7, wherein the second polymer is a biodegradable polymer.

Aspect 9. The surgical mesh of aspect 6, wherein the first polymer is polypropylene and wherein the second polymer is a poly(hydroxy acid).

Aspect 10. The surgical mesh of aspect 9, wherein the second polymer is poly(lactide-co-glycolide).

Aspect 11. The surgical mesh of aspect 6, wherein the first polymer is polypropylene and wherein the second polymer is a styrene-isobutylene copolymer.

Aspect 12. The surgical mesh of aspect 1, wherein the mesh has a pore size ranging from 0.5 to 10 mm and a filament diameter ranging from 50 to 500 μm.

Aspect 13. The surgical mesh of aspect 1, wherein the surgical mesh is a pelvic floor repair mesh.

Aspect 14. The surgical mesh of aspect 13, wherein the surgical mesh comprises a mesh body and two or more mesh arms extending from said body.

Aspect 15. The surgical mesh of aspect 3, comprising less than 1 wt % N,N-dimethyl formamide and less than 1 wt % tetrahydrofuran.

Aspect 16. A method of forming the surgical mesh of aspect 1, comprising applying a coating formulation that comprises poly(lactide-co-glycolide), minocycline and rifampin to a polypropylene mesh.

Aspect 17. The method of aspect 16, wherein said coating formulation further comprises N,N-dimethyl formamide and tetrahydrofuran.

Aspect 18. The mesh of aspect 1, which is sterile, disposed in a package that maintains the sterility of the mesh.

Aspect 19. A surgical method comprising implanting the mesh of aspect 1 into a subject.

### Example

Polypropylene mesh (filament diameter 0.5 mm) is spray coated with a coating material that contains 50 wt % PLGA (85/15 lactide/glycolide molar ratio), 33 wt % rifampin and 17 wt % minocycline. The mesh is covered to a total antimicrobial (rifampin+minocycline) dose density of 25 g/m². The mesh can be stretched on a frame, if desired, to provide support for the mesh during coating applications. The coating material is dissolved at a concentration of about 2-4 wt % solids in a solvent system that contains 50 wt % N,N-dimethylformamide (DMF) and 50 wt % tetrahydrofuran (THF). The DMF provides good solubility for the solids, while the THF (which has a lower boiling point) provides for relatively rapid evaporation upon application to the mesh. The mesh is spray coated using a conventional stent sprayer that utilizes high-speed atomized aerosol droplets which impact, spread and dry into a thin-film coating on the filaments, without filling the mesh pores. The mesh is coated with a fine spray (e.g., mean droplet size of 1-10 microns mean) at a mean spray velocity of about 20-40 m/s and at a spray-head-to-mesh distance of about 35-100 mm. Multiple passes may be employed to provide the desired loading. The mesh may be coated (e.g., by 1-4 passes), followed by convection oven drying at 65° C. for 15 minutes and repeated until coat weight is met. The mesh is then sterilized and packaged.

Although various embodiments are specifically illustrated and described herein, it will be appreciated that modifications and variations of the present invention are covered by the above teachings and are within the purview of any appended claims without departing from the spirit and intended scope of the invention.

What is claimed is:

1. A surgical mesh comprising at least one antimicrobial agent in an amount sufficient to reduce the risk of infection of the mesh by Staphylococcus aureus.

2. The surgical mesh of claim 1, wherein the at least one antimicrobial agent comprises a rifamycin group antibiotic, a tetracycline antibiotic, or a combination thereof.

3. The surgical mesh of claim 1, wherein the at least one antimicrobial agent comprises minocycline and rifampin as antibiotics.

4. The surgical mesh of claim 3, wherein aminocycline:rifampin weight ratio ranges from 1:10 to 2:1.

5. The surgical mesh of claim 3, wherein the total antibiotic concentration ranges from 5 to 100 grams of antibiotics per square meter of mesh.

6. The surgical mesh of claim 1, comprising a core comprising a first polymer and a coating comprising a second polymer and said at least one antimicrobial agent.

7. The surgical mesh of claim 6, wherein the first polymer is a biostable polymer.

8. The surgical mesh of claim 7, wherein the second polymer is a biodegradable polymer.

9. The surgical mesh of claim 6, wherein the first polymer is polypropylene and wherein the second polymer is a poly(hydroxy acid).

10. The surgical mesh of claim 9, wherein the second polymer is poly(lactide-co-glycolide).

11. The surgical mesh of claim 6, wherein the first polymer is polypropylene and wherein the second polymer is a styrene-isobutylene copolymer.

12. The surgical mesh of claim 1, wherein the mesh has a pore size ranging from 0.5 to 10 mm and a filament diameter ranging from 50 to 500 μm.

13. The surgical mesh of claim 1, wherein the surgical mesh is a pelvic floor repair mesh.

14. The surgical mesh of claim 13, wherein the surgical mesh comprises a mesh body and two or more mesh arms extending from said body.

15. The surgical mesh of claim 3, comprising less than 1 wt % N,N-dimethyl formamide and less than 1 wt % tetrahydrofuran.

16. A method of forming a surgical mesh that comprises at least one antimicrobial agent in an amount sufficient to reduce the risk of infection of the mesh by Staphylococcus aureus, said method comprising applying a coating formulation that comprises poly(lactide-co-glycolide), minocycline and rifampin to a polypropylene mesh.

17. The method of claim 16, wherein said coating formulation further comprises N,N-dimethyl formamide and tetrahydrofuran.

18. The mesh of claim 1, which is sterile, disposed in a package that maintains the sterility of the mesh.

19. A surgical method comprising implanting into a subject a surgical mesh that comprises at least one antimicrobial agent in an amount sufficient to reduce the risk of infection of the mesh by Staphylococcus aureus.
